(19) United States
(12) Patent Application Publication
(10) Pub. No.: US 2010/0210017 A1
(43) Pub. Date: Aug. 19, 2010

(54) COMPOSITIONS AND METHODS FOR ENHANCING TOLERANCE FOR THE PRODUCTION OF ORGANIC CHEMICALS PRODUCED BY MICROORGANISMS

(76) Inventors: Ryan T. Gill, Denver, CO (US); Tanya E. Lipscomb, Boulder, CO (US); Michael D. Lynch, Boulder, CO (US)

Correspondence Address: WILSON, SONSINI, GOODRICH & ROSATI, 650 PAGE MILL ROAD, PALO ALTO, CA, 94304-1050, US

(21) Appl. No.: 12/523,047

(22) PCT Filed: Jan. 11, 2008
(86) PCT No.: PCT/US08/50921

§ 371 Date: Apr. 23, 2010

Related U.S. Application Data
(60) Provisional application No. 60/880,108, filed on Jan. 12, 2007.

(51) Int. Cl. C12N 15/70; C12N 1/00; C12N 1/21; C12N 15/00

(52) U.S. Cl. 435/471; 435/243; 435/252.33

(57) ABSTRACT

Embodiments herein generally relate to methods, compositions and uses for enhancing tolerance of production of organic acids and alcohols by microorganisms. This application also relates generally to methods, compositions and uses of vectors having one or more genetic element to increase the tolerance of organic acids or alcohols by a microorganism. Certain embodiments relate to compositions and methods of enhancing the tolerance for production of 3-hydroxypropionic acid (3-HP) by bacteria. In some embodiments, compositions and methods relate to regulating the expression of an inhibitory molecule of an enhancing gene to increase production of organic acid by bacteria.

### RELATED APPLICATIONS

This application claims the benefit under 35 U.S.C. §119(e) of U.S. provisional patent application Ser. No. 60/880,108 filed on Jan. 12, 2007, incorporated herein by reference in its entirety.

### FEDERALLY FUNDED RESEARCH

Embodiments disclosed herein were supported in part by grant BES0228584 from the National Science Foundation. The U.S. government may have certain rights to practice the subject invention.

### FIELD

Embodiments herein generally relate to methods, compositions and uses for enhancing tolerance of and/or production of organic acids and alcohols by microorganisms. This application also relates generally to methods, compositions and uses of vectors to increase the production of organic acids or alcohols by a microorganism. Certain embodiments relate to compositions and methods of enhancing the tolerance to 3-hydroxypropionic acid as a means to increase production of 3-hydroxypropionic acid (3-HP) by bacteria. In other embodiments, compositions and methods relate to regulating one or more inhibitory molecules or enhancing molecules of a chorismate super-pathway of a microorganism to increase tolerance to production of organic acid by the microorganism.

### BACKGROUND

Oil costs have risen dramatically over the past several years. Most experts now believe that such cost increases will continue and that oil production capacity will peak in the near future. Alternative sources of inexpensive materials and energy for the production of fuels and other chemicals must be developed. Biorefining seeks to develop renewable resources, such as agricultural or municipal waste, for such purposes. The basic model involves the conversion of waste material (e.g. corn) into sugars (e.g. hexoses, pentoses) that can be fermented by engineered organisms to produce value added products such as fuels (e.g., ethanol or hydrogen) or commodity chemicals (e.g. monomers/polymers). While much debate still exists regarding the long term commercial viability of ethanol as a gasoline replacement, biological routes for the production of commodity chemicals have been proven as economically attractive alternatives to conventional petrochemical routes. As one example, a decade long Dupont/Genencor collaboration led Dupont into investing in the development of an 800,000 liters E. coli based process for the production of 1,3 propanediol (an estimated $5-8 billion/year product).

Organic acids represent an important platform of future biorefining chemicals. In a report released by the National Renewable Energy Laboratory, eight different organic-acids were ranked among the top 12 highest priority biorefining chemicals that include 3-hydroxypropionic acid (3-HP). There remains a need for rapidly generating these biorefining chemicals in low cost efficient methods.

### SUMMARY

Embodiments herein concern methods and compositions for increasing tolerance to organic compound production by microorganisms. Certain embodiments, concern increasing tolerance for biorefining chemicals. In other embodiments, compositions and methods herein concern production of 3-hydroxypropionic acid (3-HP). Microorganisms contemplated of use herein can include, but are not limited to, E. coli.

Products of the pathway can include, but are not limited to, one or more of chorismate, tyrosine, phenylalanine, tryptophan, folate, ubiquinone, meniquinone, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8 or ubiquinone-8,3-deoxy-D-arbino-heptulosonate-7-phosphate synthase (DAHPS) isozymes, or a mixture thereof.

Some embodiments concern composition for increasing the tolerance of 3-HP production by a microorganism including a vector having one or more genetic elements capable of modulating the chorismate super-pathway of the microorganism wherein modulation of the chorismate super-pathway increases the tolerance of 3-HP by the microorganism. In other embodiments, the composition may include intermediates of the chorismate super-pathway. In yet other embodiments, the composition may include one or more products or precursors of the pathway.

Products of the pathway can include, but are not limited to, one or more of chorismate, tyrosine, phenylalanine, tryptophan, folate, ubiquinone, meniquinone, 3-deoxy-D-arbino-heptulosonate-7-phosphate synthase (DAHPS) isozymes, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8 or ubiquinone-8.

Other embodiments herein include compositions for increasing tolerance of 3-hydroxypropionic acid (3-HP) production by a microorganism including, but not limited to, one or more compounds capable of modulating chorismate super-pathways of the microorganism wherein induction of the chorismate super-pathways increase the production of 3-HP by the microorganism. In accordance with these embodiments compositions can include, but are not limited to, one or more intermediates, or compositions capable of increasing and/or decreasing production of one or more intermediates, of the chorismate super-pathway. Other compositions, can include one or more precursors, or compositions for increasing and/or decreasing production of one or more precursors to the chorismate super-pathway. Some embodiments can further include, but are not limited to, one or more compounds chosen from one or more of chorismate, tyrosine, phenylalanine, tryptophan, folate, ubiquinone, meniquinone, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8 or ubiquinone-8,3-deoxy-D-arbino-heptulosonate-7-phosphate synthase (DAHPS) isozymes, or a mixture thereof. Other embodiments may include compounds that induce one or more enzymes of the chorismate super-pathway in the microorganism. Other exemplary compounds can include one or more vectors capable of modulating the chorismate super-pathway introduced to an organic acid-producing microorganism.

Some embodiments include compositions for modulating tolerance for production of 3-hydroxypropionic acid (3-HP) by a microorganism including; one or more compounds capable of modulating the chorismate super-pathway by the microorganism wherein induction of the chorismate super-pathway increases tolerance of 3-HP by the microorganism.

Certain embodiments herein concern compositions including, but not limited to one or more compounds, including, but not limited to, chorismate, tyrosine, phenylalanine, tryptophan, folate, ubiquinone, meniquinone, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8 or ubiquinone-8,3-deoxy-D-arbino-heptulosonate-7-phosphate synthase (DAHPS) isozymes, or a mixture thereof.

Other embodiments herein include methods for increasing tolerance for production of an organic acid by a microorganism including, but not limited to, inhibiting repressors capable of affecting the chorismate super-pathway in the microorganism. In accordance with these embodiments, other compounds capable of increasing production of or tolerance for organic acids or alcohol may be combined, or added separately to any culture contemplated herein. In addition, it is contemplated herein that methods and compositions disclosed may be used in combination with other known 3-HP production technologies known in the art.

In accordance with any of these embodiments, one or more compounds and or compositions can be introduced to a microorganism wherein the compound and/or composition is capable of modulating the chorismate super-pathway and increasing tolerance of the microorganism to 3-HP production. In addition, it is contemplated herein that methods and compositions herein can be combined with any other method known to increase the tolerance for or production of an organic acid in a microorganism.

Some embodiments can include methods for increasing the production of and/or tolerance for production of an organic acid by a microorganism comprising: a) obtaining one or more compounds capable of modulating aspects of chorismate super-pathway by the microorganism. In certain embodiment, modulation of the chorismate super-pathways increases the tolerance for 3-HP production by the microorganism; and b) introducing the compounds to a culture of the microorganism.

Certain embodiments herein concern the production or increased tolerance for the organic acid, 3-HP. In accordance with these embodiments, one or more compounds contemplated herein to increase the tolerance for or production of 3-HP can include, but are not limited to, the composition comprises one or more intermediate of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

Yet other embodiments herein include methods for increasing the production of an organic acid such as, 3-hydroxypropionic acid (3-HP), by a microorganism comprising contacting a culture of microorganism with a composition comprising one or more compounds of chorismate super-pathways and/or one or more compounds capable of modulating the chorismate super-pathways. In accordance with these embodiments, one or more compounds can include a vector having one or more genetic elements capable of modulating, such as increasing or decreasing the chorismate super-pathway. Some embodiments contemplated herein are directed towards the use of other compounds, these compounds can include a vector having one or more genetic element capable of increasing downstream components for the chorismate super-pathway to increase tolerance for 3-HP in a microorganism.

In some embodiments, methods for increasing the production and/or tolerance of 3-hydroxypropionic acid (3-HP) by a microorganism can include, but are not limited to, genetically manipulating chorismate super-pathways in the microorganism. Some of these genetic manipulations of the chorismate super-pathway in a microorganism are chosen from modulating the chorismate super-pathway in a microorganism by adding a vector to introduce new genetic material; genetic insertion, disruption or removal of existing genetic material; mutation of genetic material and a combination thereof. Genetic manipulations can include the induction of one or more of a chorismate super-pathway precursor, chorismate, tyrosine, phenylalanine, tryptophan, folate, ubiquinone, meniquinone, 3-deoxy-D-arbino-heptulosonate-7-phosphate synthase (DAHPS) isozymes, shikimate, or a mixture thereof.

Some embodiments herein may be combined with other methods or compositions known in the art to increase tolerance for organic acid production in a microorganism. In other embodiments, methods and compositions herein may be combined with strain selection processes in order to identify strains capable of producing and/or tolerating increased concentrations of 3-HP. For example, as referenced herein, Multi-Scale Analysis of Library Enrichments (SCALEs) can be used to identify genes conferring increased fitness in continuous flow selections. These selections may be based on the presence or absence of a selective compound such as one or more organic acids or alcohols of interest. Some embodiments concern selection with increasing organic acid, for example, 3-hydroxypropionic acid (3-HP) at inhibitory levels. These selection processes can be based on SCALES alone or in combination with other selection technologies, for example, other genomic selection technologies.

In certain embodiments, kits are contemplated herein. In certain embodiments, a kit for increasing production of an organic acid in a microorganism can include, but is not limited to, one or more compounds capable of modulating chorismate super-pathway; and one or more containers. In accordance with these embodiments, a kit can include one or more compounds is chosen from chorismate, tyrosine, phenylalanine, tryptophan, folate, ubiquinone, meniquinone, 3-deoxy-D-arbino-heptulosonate-7-phosphate synthase (DAHPS) isozymes, shikimate, precursor of the chorismate super-pathway, one or more enzymes of the chorismate super-pathway D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

In certain embodiments, a kit for increasing production of an organic acid in a microorganism can include, but is not limited to, one or more compounds capable of modulating chorismate super-pathway where modulation concerns intracellular levels of one or more intermediate of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

The skilled artisan will realize that although methods and compositions are described in terms of embodiments for application of increasing tolerance for 3-HP production in microorganisms, they may also of use with other types of organic acid tolerance in microorganisms.

### DESCRIPTION OF ILLUSTRATIVE EMBODIMENTS

The following drawings form part of the present specification and are included to further demonstrate certain embodiments. The embodiments may be better understood by reference to one or more of these drawings in combination with the detailed description of specific embodiments presented herein.

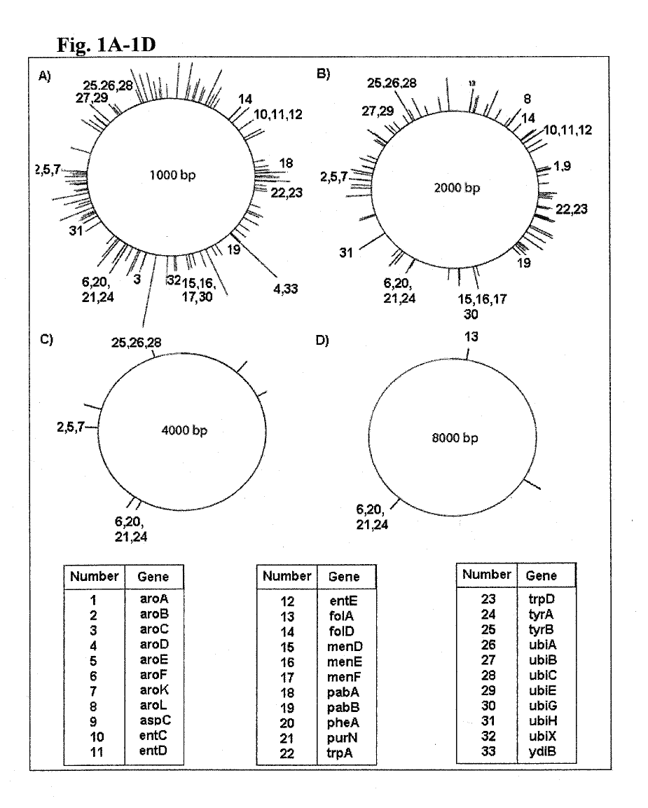

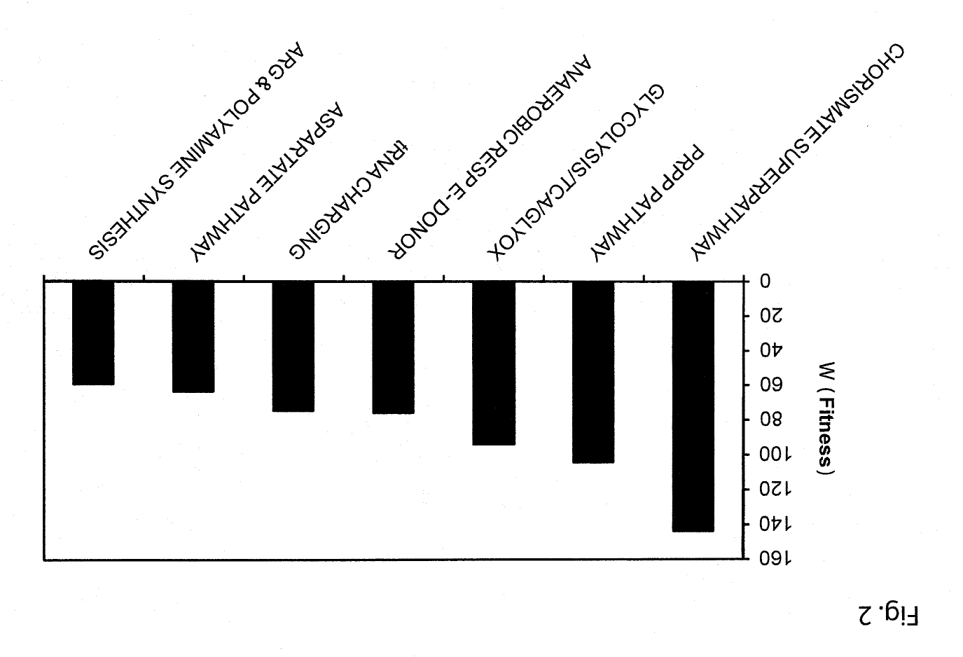

FIGS. 1A-1D represent schematics of genome-wide multiscale analysis from 3-HP selection. A) represents signal associated with the 1000 base pair scale (bp); B) represents signal associated with the 2000 bp scale, C) represents signal associated with the 4000 bp scale and D) represents signal associated with the greater than 8000 bp scale

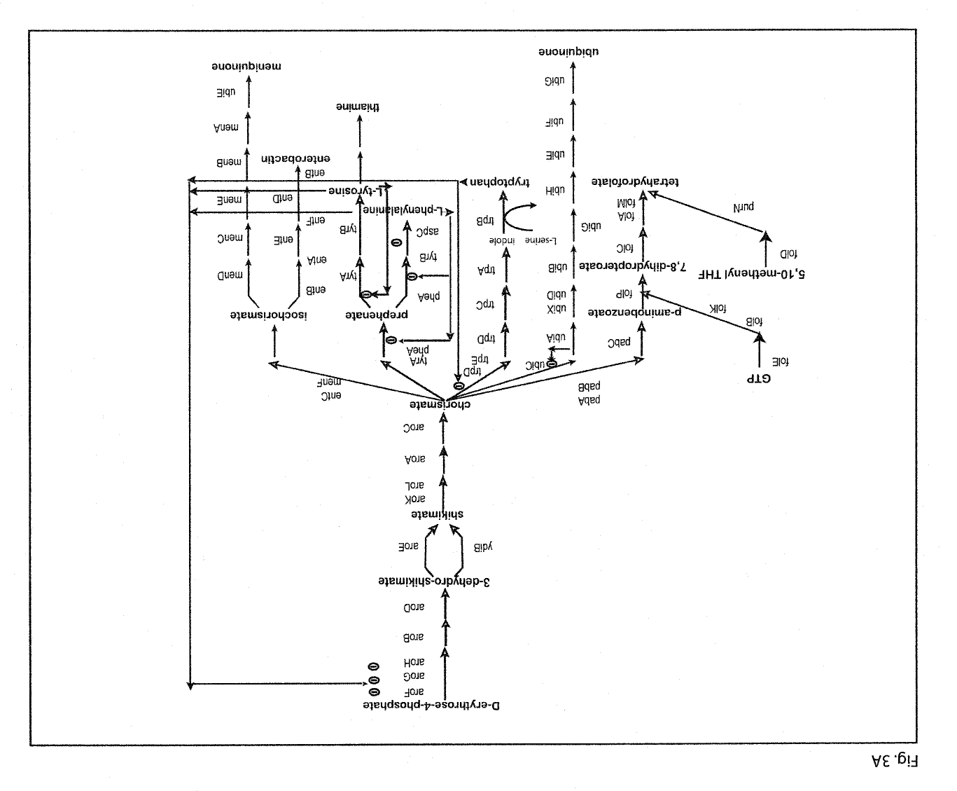

FIG. 2A represents an exemplary histogram plot of seven pathways contributing to fitness in the presence of 3-HP.

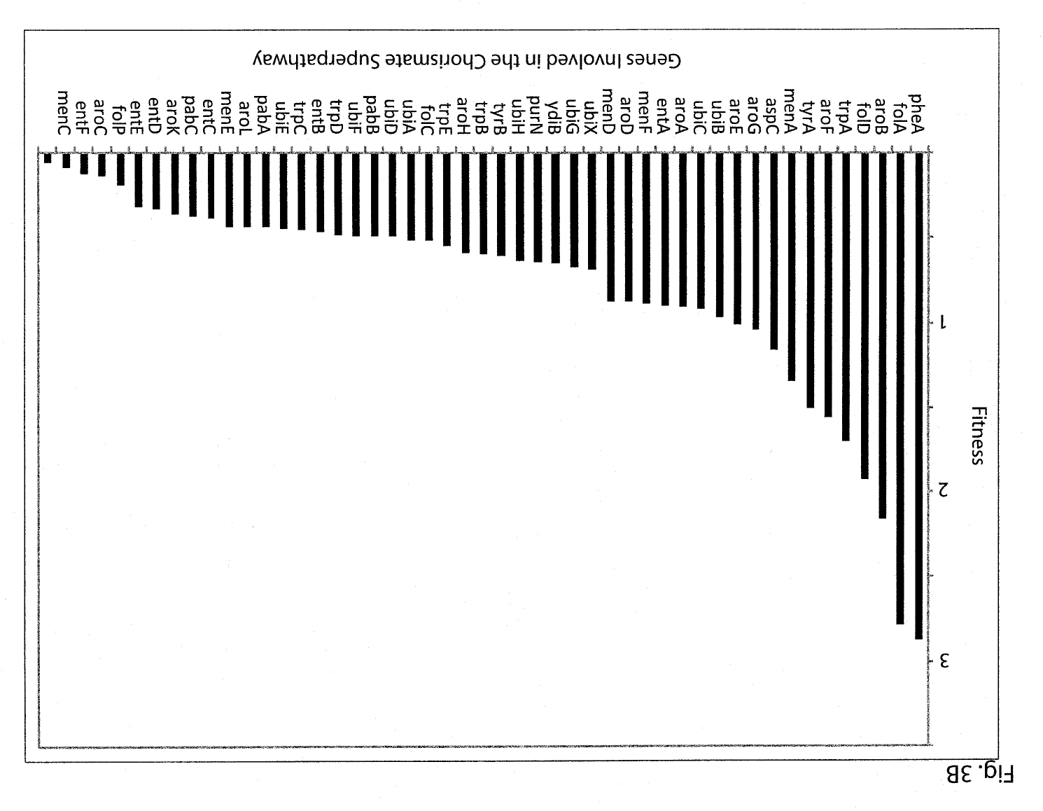

FIG. 3A represents an exemplary schematic of a chorismate super-pathway.

FIG. 3B represents exemplary bar graph of change in fitness (increase in growth rate) associated with increase in copy number of chorismate super-pathway-associated genes as designated.

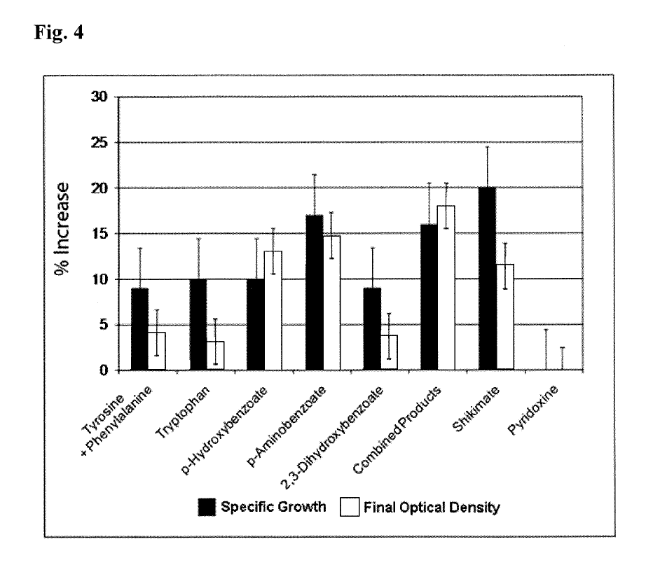

FIG. 4 represents an exemplary bar graph of growth of microorganisms in the presence or absence of exogenously added organic molecules or combinations of molecules.

### DEFINITIONS

As used herein, “a” or “an” may mean one or more than one of an item.

As used herein, “modulate” or “modulating” or “modulation” may mean altering, increasing or decreasing.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times, temperature and other specific details may be modified through routine experimentation. In some cases, well known methods or components have not been included in the description.

In accordance with embodiments herein, there may be employed conventional molecular biology, microbiology, and recombinant DNA techniques within the skill of the art. Such techniques are explained fully in the literature. (See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Biorefining concerns the development of efficient processes for the conversion of renewable sources of carbon and energy into large volume commodity chemicals. The US Department of Energy (USDOE) has publicized a prioritized list of building block chemicals for future biorefining endeavors, which includes for example, 3-hydroxypropionic acid (3-HP). Previous production was accomplished by development of recombinant hosts that convert glucose to 3-HP. It has been proposed that final 3-HP titers of at least 100 g/L are needed to ensure economic feasibility for industrial production, but as low as 10 g/L in these cultures can inhibit growth

Several different genetic strategies have been investigated for the production of 3-HP in E. coli, which is an attractive host organism because of its large nutrient source range (e.g. pentoses), fast growth, and ability to be easily genetically modified when compared to alternative organisms. One issue has been low tolerance for high level production of organic compounds by a microorganism. Often, the increased organic compound becomes toxic to the microorganism. A need exists for improving the production of and tolerance for organic acid and alcohol production by microorganisms.

Scalar Analysis of Library Enrichment (SCALEs), is a high-resolution, genome-wide approach that can be used to monitor enrichment and dilution of individual clones within a genomic-library population. This method includes creation of representative genomic libraries with varying insert size, growth of clones in selective environments, interrogation of the selected population using microarrays, and a mathematical multi-scale analysis to identify the gene(s) for which increased copy number improves overall fitness. This method has been employed to develop the technique of directed strain selection relevant for organic acid phenotypes, for example, 3-HP tolerance phenotypes (data not shown). Previous work has identified several mechanisms of alleviating product toxicity including: biofilm formation, altered permeability, increased transport, product modification or carbon utilization, and specific metabolic changes. In certain embodiments, methods herein seek to evaluate the inhibition due to metabolic effects specific to organic acid stress, for example, 3-HP stress, within the cell related to the chorismate biosynthetic pathway.

Certain embodiments concern biorefining, biomass (e.g. crops, trees, grasses, crop residues, forest residues) and using biological conversion, fermentation, chemical conversion and catalysis to generate and use organic compounds. These organic compounds can then subsequently be converted to valuable derivative chemicals. However, the organic acids can be toxic by nature and thus inhibitory to the production organisms at low levels. In order to optimize production of the organic acid intermediates, engineering tolerance to the organic acid may be a factor. This can be accomplished by supplying exogenous molecules to enhance production or to inhibit expression of a non-permissive molecule thereby permitting increased levels of production. Since commodity chemicals exist in a competitive environment, optimization might be necessary for the economic feasibility of biorefining. Therefore, compositions and methods disclosed herein are directed toward identifying bacterial strains and genetic regions within molecules that increase production of or tolerance to organic compounds for use in bioproduction products and systems.

### Chorismate Super-Pathway

The chorismate super-pathway is a primary metabolic pathway essential for cell viability. For example, chorismate is the common precursor to a number of aromatic amino acids (tyrosine, phenylalanine, tryptophan) and vitamins (folate, ubiquinone, and meniquinone) required for cell viability. In one more particular example, 3-deoxy-D-arbino-heptulosonate-7-phosphate synthase (DAHPS) isozymes active in the first step of chorismate synthesis (aroF, aroG, aroH) show significant feedback inhibition from increased aromatic amino acid pools produced downstream. In one embodiment herein, the chorismate super-pathway can be inhibited by 3-HP stress, which can be partially alleviated by the addition of a downstream product of the chorismate super-pathway to the growth media. In one more particular embodiment, the downstream product can be shikimate. Addition of each downstream product from chorismate shows at least a partial regeneration of specific growth and final cell density. In one particular embodiment, addition of shikimate can lead to about 20% regeneration of growth compared with wild-type growth, indicating that inhibition may occur prior to the formation of shikimate, leading to a reduced amino acid and vitamin pool within the cell.

In various embodiments, growth can be enhanced by identifying a gene that with modulated expression can increase the tolerance and/or production of an organic compound. In some embodiments, modulation can include an increase in expression or activity of one or more genes of the chorismate super-pathway. In other embodiments, modulation can include a decrease in expression or activity of one or more genes of the chorismate super-pathway. In other embodiments, modulation of the chorismate super-pathway can include a combination of increasing the expression and/or activity of some genes while decreasing the expression and/or activity of other genes. In some embodiments, genes capable of altering the chorismate super-pathway can include genes that alter the formation of an intermediate of the pathway and/or alter precursors of the pathway. It is contemplated herein that genetic manipulation can include, increasing and/or decreasing flux of intermediates through the chorismate super-pathway.

Genetic screens, used to detect individual compounds, often proceed one cell at a time. Selections are tied to viability in a specific environment. Therefore, in one embodiment, bacterial organisms that demonstrate increased growth or tolerance for an organic acid may be selected for and the genetic region that affects growth, production and/or tolerance identified. In some embodiments, selection of a genetic region encoding tyrosine demonstrated increased production of and/or tolerance of an organic acid molecule produced in a bacteria.

Certain embodiments herein concern modulating the chorismate super-pathway capable of enhancing tolerance of organic compound production in a microorganism. In accordance with these embodiments, expression of certain molecules within this pathway is capable of increasing tolerance of an organic compound by modulating the expression of genes of the pathway. This novel tolerance strategy will allow increased production of organic compounds, such as 3-HP. For example, strains already engineered to produce 3-HP can be modified by modulating one or more genes in the chorismate super-pathway disclosed herein to increase tolerance of the strain to produce 3-HP. In addition, these methods may be used in conjunction with the SCALEs technology (U.S. Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” incorporated herein by reference in their entirety), for genetic alterations of organisms and for genetic selection strategies.

In some embodiments, genetic manipulation of microorganisms can de used to make desired genetic changes that can result in desired phenotypes and can be accomplished through numerous techniques. These techniques include, but are not limited to, using: i) a vector to introduce new genetic material; ii) genetic insertion, disruption or removal of existing genetic material, as well as; iii) mutation of genetic material; or any combinations of i, ii, and iii, that results in desired genetic changes with desired phenotypic sought. A vector can include, but is not limited to, any genetic element used to introduce new genetic material into an organism. These vectors can include, but are not limited to, a plasmid of any copy number, an integratable element that integrate at any copy into the genome, a virus, phage or phagemid. In other embodiments herein, genetic insertions, disruptions or removals can be included as part of inserting a new genetic element into the genome, disruption transcription or normal regulatory function via insertion that can affect larger regions of the genome in addition to those at the site of insertion, and the deletion or removal of a region of the genome. These can be done with techniques including, but not limited to, directed knock-outs or mutations, gene replacements, transposons, random mutagenesis or a combination thereof. Mutations can be directed or random, utilizing any techniques requiring vectors, insertions, disruptions or removals, in addition to those including, but not limited to, error prone or directed mutagenesis through PCR, mutator strains, and random mutagenesis, by any technique known in the art.

In certain embodiments, SCALEs can be used to monitor enrichment and dilution of individual clones within a genomic-library population. This method includes creation of representative genomic libraries with varying insert size, growth of clones in selective environments, interrogation of the selected population using microarrays, and a mathematical multi-scale analysis to identify the gene(s) for which increased copy number improves overall fitness.

In addition, certain embodiments contemplated herein relate to inhibiting the expression or activity of a repressor gene corresponding to an enhancing gene (e.g. a gene that increases production or increases tolerance of production of an organic acid by a microorganism). In other embodiments, clones carrying a deletion in the TyrR region (tyrosine repressor gene region), the repressor region corresponding to the Tyrosine and Chorismate pathways, can be used to increase tyrosine pools. Combination of this repressor with other chorismate pathway mutations could result in alteration of intermediate pools related to increased shikimate production and corresponding increased 3-HP tolerance. In certain embodiments, a genetic region equivalent to, corresponding to or including about 50%, or about 60%, or about 70%, or even about 80% or about 90% of the gene region spanning from 2736799-2738100 (Tyrosine A clone) in MACH1 cultures and/or gene region spanning from 2736700-2739223 (Tyrosine A clone) can be used herein to increase the production of or tolerance for production of 3-HP by a microorganism. In addition, it is contemplated herein that a mutation/deletion within a genetic region equivalent to, corresponding to or including about 50%, about 60%, about 70%, about 80% or about 90% of the gene region spanning from 1384744-1386285 (Tyrosine R clone) can be used herein to increase the production of or tolerance for 3-HP production by a microorganism. In one embodiment, one or more mutation/deletion may be within a genetic region encoding a repressor capable of repressing any amino acid produced in the chorismate super-pathway, for example, tyrosine. Note: the percentage contemplated herein may include non-contiguous regions.

In one exemplary method, pathway fitness analysis identified multiple pathways, each of which play a role in growth inhibition specific to increased levels of 3-HP, including the chorismate super-pathway and the histidine, purine, and pyrimidine biosynthesis super-pathway (PRPP) (see for example, FIG. 2). This genome-wide, quantitative methodology has enabled us to identify entire metabolic pathways associated with growth inhibition due to 3-HP stress

Some embodiments concern compositions for increasing the tolerance for 3-hydroxypropionic acid (3-HP) by a microorganism comprising; one or more compounds capable of modulating chorismate super-pathway of the microorganism wherein modulation of the chorismate super-pathway increases the tolerance of 3-HP. In certain embodiments, the composition includes an intermediate of the chorismate super-pathway. In other embodiments, the composition includes a precursor to the chorismate super-pathway. In yet other embodiments, the composition includes modulating flux of the chorismate super-pathway. In some embodiments, modulate can mean increase or decrease expression or activity of one or more genes of the chorismate super-pathway. In accordance with these embodiments, one or more compounds can induce an enzyme of the chorismate super-pathway in the microorganism. In other embodiments, the compound can include a vector having a genetic element capable of modulating the chorismate super-pathway.

Compositions and methods of use contemplated herein can include, but are not limited to, one or more intermediate of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy-1,4-benzoquinone, 3-demethylubiquinone-8, ubiquinone-8 or a combination, or mixture of two or more thereof.

Compositions and methods of use contemplated herein can include, but are not limited to, one or more precursor of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy-1,4-benzoquinone, 3-demethylubiquinone-8, ubiquinone-8 and a combination, or mixture of, two or more thereof.

Compositions and methods of use contemplated herein can include, but are not limited to, one or more composition that is capable of altering intracellular levels of one or more intermediate of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

Compositions and methods of use contemplated herein can include, but are not limited to, one or more composition capable of altering intracellular levels of one or more precursors of the chorismate super-pathway chosen from D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

Compositions and methods of use contemplated herein can include, but are not limited to, one or more compound chosen from chorismate, tyrosine, phenylalanine, tryptophan, folate, ubiquinone, meniquinone, 3-deoxy-D-arbino-heptulosonate-7-phosphate synthase (DAHPS) isozymes, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8 or ubiquinone-8.

In some embodiments, compositions and methods of use herein can concern use of a compound that modulates one or more enzymes of the chorismate super-pathway in the microorganism. In certain embodiments, compositions and methods of use herein can concern use of a compound that modulates one or more the compound by introducing one or more vector s having genetic element(s) capable of altering metabolites of the chorismate super-pathway. In certain embodiments, compositions and methods of use herein can concern one or more compound(s) capable of modulating a genetic change that alters metabolites in the chorismate super-pathway.

Other embodiments concern compositions or methods of use for increasing the production of 3-hydroxypropionic acid (3-HP) by a microorganism using one or more compounds capable of increasing the tolerance of the microorganism to 3-HP, wherein the composition induces tolerance to at least 30 g/L of 3-HP. Other embodiments contemplated included tolerance to at least 35 g/L of 3-HP; to at least 40 g/L 3-HP; to at least 1.2 fold 3-HP of a wild-type composition, to at least 1.4 fold 3-HP of a wild-type composition; to at least 1.6 fold 3-HP of a wild-type composition, where the wild-type composition has little or no chorismate super-pathway altering compositions or methods.

Other exemplary methods contemplated herein concern increasing the production of or tolerance for production of an organic acid by a microorganism comprising, modulating the chorismate super-pathway in the microorganism. In accordance with these exemplary methods, modulating the chorismate super-pathway in the microorganism can include introducing a compound to the microorganism capable of modulating the chorismate super-pathway. Other methods contemplated for increasing the production of or tolerance for production of an organic acid by a microorganism can include: obtaining one or more compounds capable of modulating intermediates of chorismate super-pathways by the microorganism wherein modulation of the chorismate super-pathways increases the production of or tolerance for the organic acid by the microorganism; and introducing the compounds to a culture of the microorganism. In certain more particular embodiments the organic acid is 3-HP or a 3-HP composition.

In some embodiments, compounds can be chosen from one or more of chorismate, tyrosine, phenylalanine, tryptophan, folate, ubiquinone, meniquinone, 3-deoxy-D-arbino-heptulosonate-7-phosphate synthase (DAHPS) isozymes, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy-1,4-benzoquinone, 3-demethylubiquinone-8, ubiquinone-8 or a combination of, or mixture of two or more thereof.

In some embodiments contemplated herein, compositions of 3-HP can contain a mixture of 3-HP, and optionally, one or more of 3,3-dioxproprinic acid and acrylic acid.

Some exemplary methods contemplated herein concern increasing production of or tolerance for production of an organic acid by a microorganism including: obtaining one or more compounds capable of modulating precursors of chorismate super-pathways by the microorganism wherein induction of the chorismate super-pathways increases the production of or tolerance for the organic acid by the microorganism; and introducing the compounds to a culture of the microorganism.

In some more particular methods, increasing the production of 3-hydroxypropionic acid (3-HP) by a microorganism can include contacting a culture of microorganism with a composition comprising one or more compounds of chorismate super-pathway or capable of modulating the chorismate super-pathway. In accordance with these embodiments the compound can include a vector containing a genetic element capable of modulating the chorismate super-pathway. Other exemplary methods for increasing the production and/or tolerance of 3-hydroxypropionic acid (3-HP) by a microorganism can include genetically manipulating chorismate super-pathways in the microorganism. Genetic manipulation of the chorismate super-pathway as contemplated herein can include altering gene expression of one or more genes involved in the chorismate super-pathway in a microorganism by adding a vector to introduce new genetic material; genetic insertion, disruption or removal of existing genetic material; mutation of genetic material or a combination of two or more thereof.

Exemplary genetic insertions can include modulating intracellular levels of one or more of chorismate, tyrosine, phenylalanine, tryptophan, folate, ubiquinone, meniquinone, 3-deoxy-D-arbino-heptulosonate-7-phosphate synthase (DAHPS) isozymes, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

In some embodiments, kits are contemplated of use for compositions and methods of use contemplated herein. Certain embodiments include kits for increasing production of an organic acid in a microorganism comprising; one or more compounds capable of modulating chorismate super-pathways; and one or more containers. In accordance with these embodiments, kits of use herein can provide chorismate super-pathway altering or supplementary compositions capable altering the flux of the chorismate super-pathway in a microorganism of use for producing 3-HP. Certain embodiments can include, but are not limited to, one or more compounds is chosen from chorismate, tyrosine, phenylalanine, tryptophan, folate, ubiquinone, meniquinone, 3-deoxy-D-arbino-heptulosonate-7-phosphate synthase (DAHPS) isozymes, shikimate, D-Erythrose-4-phosphate, 3-deoxy-D-arabino-heptulosonate-7-phosphate, 3-dehydroquinate, 3-dehydro-shikimate, shikimate, shikimate-3-phosphate, 5-enolpyruvyl-shikimate-3-phosphate, chorismate, isochorismate, prephenate, phenylpyruvate, para-hydroxyphenylpyruvate, L-phenylalanine, L-tyrosine, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, L-tryptophan, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy1,4-benzoquinone, 3-demethylubiquinone-8, ubiquinone-8 and a combination, or mixture of, two or more thereof.

In some embodiments contemplated herein, compositions can include a composition capable of modulating flux of metabolites through the chorismate super-pathway, to increase and/or decrease metabolite production through the pathway. In certain examples, increase in flux can be from D-erythrose-4-phosphate to shikimate; and/or from shikimate to chorismate; and/or from chorismate to para-aminobenzoate; and/or from chorismate to ubiquinone; and/or from chorismate to tryptophan; and/or from chorismate to prephenate; and/or from chorismate to isochorismate; and/or from to para-aminobenzoate to tetrahydrofolate; and/or from prephenate to L-phenylalanine; and/or from prephenate to Tyrosine; and/or from isochorismate to enterobactin from isochorismate to meniquinone; and/or from tyrosine to thiamine

In some embodiments, genetic manipulations can be carried out to alter the intracellular concentrations of intermediates in the chorismate super pathway. In accordance with these embodiments, this pathway can be feedback inhibited causing a decrease in one or more particular intermediates that may be predicted to cause a decrease in feedback inhibition and thereby increase the flux through the chorismate super-pathway and availability of the downstream products which have been shown to increase tolerance to 3-HP. In certain embodiments, genetic manipulation may be used to reduce the amount of an intermediate of the chorismate super-pathway and this reduction may lead to an increase in tolerance of 3-HP by microorganisms

It is contemplated that one or more genes of the chorismate super-pathway used in methods and compositions herein may include all or part of the gene in order to modulate the pathway. For example, perhaps 30 percent of a gene or greater, 50 percent of a gene or greater, 70 percent of a gene or greater, or 80 percent of a gene or greater, or 90 percent of a gene or greater, or even 100 percent of a gene or greater may be used in methods and compositions contemplated herein to increase 3-HP tolerance in a microorganism (see for example, the Tyr A gene). In certain embodiments oligonucleotides comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more contiguous nucleotides having a sequence selected from genes involved in the chorismate super-pathway are contemplated. In addition, combination methods using genetic manipulation and other tolerance inducing methods are contemplated.

3-HP tolerance is important as increased tolerance can lead to increased productivities and titers in a commercial fermentation to produce 3-Hp. The basic fermentation model involves the conversion of waste material or renewable sugar feedstock (e.g. corn) into sugars (e.g. hexoses, pentoses) that can be fermented by engineered organisms to produce value added products such as fuels (e.g., ethanol or hydrogen) or commodity chemicals (e.g. monomers/polymers) such as 3-HP. 3-HP can be converted to high value chemicals that may be of interest to the chemical industry, biotech, clothing and possibly healthcare industry including new polymers and materials, as well as traditional large market chemicals such as acrylic acid, acrylamide, methyl-acrylate, 1,3 propanediol.

### Nucleic Acids

Nucleic acids within the scope contemplated herein may be made by any technique known to one of ordinary skill in the art. Examples of nucleic acids, particularly synthetic oligonucleotides, can include a nucleic acid made by in vitro chemical synthesis using phosphotriester, phosphite or phosphoramidite chemistry and solid phase techniques via deoxynucleoside H-phosphonate intermediates. In certain embodiments, nucleic acid sequences contemplated herein can be generated and may be modified. Examples of modified nucleic acid sequences include those that can be modified after amplification reactions such as PCR™ or the synthesis of oligonucleotides. Examples of a biologically produced nucleic acids include recombinant nucleic acid production in living cells, such as recombinant DNA vector production in bacteria.

Nucleobase, nucleoside and nucleotide mimics or derivatives are well known in the art, and have been described. Purine and pyrimidine nucleobases encompass naturally occurring purines and pyrimidines and derivatives and mimics thereof. These include, but are not limited to, purines and pyrimidines substituted with one or more alkyl, carboxyalkyl, amino, hydroxyl, halogen (e.g. fluoro, chloro, bromo, or iodo), thiol, or alkylthiol groups. The alkyl substituents may comprise from about 1, 2, 3, 4, or 5, to about 6 carbon atoms.

Examples of purines and pyrimidines contemplated to modify nucleic acids produced herein can include, but are not limited to, deazapurines, 2,6-diaminopurine, 5-fluorouracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcytosine, 5-bromouracil, 5-ethyluracil, 5-iodouracil, 5-chlorouracil, 5-propyluracil, thiouracil, 2-methyladenine, methylthioadenine, N,N-dimethyladenine, azaadenines, 8-bromoadenine, 8-hydroxyadenine, 6-hydroxyaminopurine, 6-thiopurine, 4-(6-aminohexyl/cytosine), and the like. In addition, purine and pyrimidine derivatives or mimics can be used as base substitutions in any of the methods disclosed herein.

For applications in which the nucleic acid segments are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

In some embodiments, DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of a selected gene or selected gene segment.

Where an expression product is to be generated, it is possible for the nucleic acid sequence to be varied while retaining the ability to encode the same product

### Amplification

Amplification may also be of use in the iterative process for generating multiple copies of a given nucleic acid sequence. Within the scope, amplification may be accomplished by any means known in the art.

### Primers

Primer, as needed herein, are meant to encompass any nucleic acid that is capable of priming the synthesis of a nascent nucleic acid in a template-dependent process. Typically, primers are oligonucleotides around 5-100 base pairs in length, but longer sequences may be employed. Primers may be provided in double-stranded or single-stranded form.

In some embodiments, amplification of a random region is produced by mixing equimolar amounts of each nitrogenous base (A, C, G, and T) at each position to create a large number of permutations (e.g. where “n” is the oligo chain length) in a very short segment. This provides dramatically more possibilities to find high affinity nucleic acid sequences when compared to the 10.9 to 1011 variants of murine antibodies produced by a single mouse.

A number of template dependent processes are available to amplify marker sequences present in a given template sample. One of the best known amplification methods is the polymerase chain reaction (referred to as PCR) which is described in detail in U.S. Pat. Nos. 4,683,195, 4,683,202 and 4,800,159, incorporated herein by reference in their entirety.

In other embodiments, other methods for amplification of nucleic acids, include but are not limited to, the ligase chain reaction (“LCR”), Qbeta Replicase, isothermal amplification methods, and Strand Displacement Amplification (SDA), as well as other methods known in the art. Still other amplification methods may be used in accordance with embodiments disclosed herein. Other nucleic acid amplification procedures may include transcription-based amplification systems (TAS), including nucleic acid sequence based amplification (NASBA). In some of the disclosed methods, the nucleic acid sequences may be prepared for amplification by standard phenol/chloroform extraction, heat denaturation of a clinical sample, treatment with lysis buffer and mini-spin columns for isolation of DNA and RNA or guanidinium chloride extraction of RNA. In an isothermal cyclic reaction, the RNA's are reverse transcribed into double stranded DNA, and transcribed once again with a polymerase such as T7 or SP6.

Polymerases and Reverse Transcriptases include but are not limited to thermostable DNA Polymerases: OnmiBase™. Sequencing Enzyme Pfu DNA Polymerase Taq DNA Polymerase Taq DNA Polymerase, Sequencing Grade TaqBead.™. Hot Start Polymerase AmpliTaq Gold Tfl DNA Polymerase Tli DNA Polymerase Tth DNA Polymerase DNA POLYMERASES: DNA Polymerase I, Klenow Fragment, Exonuclease Minus DNA Polymerase I DNA Polymerase I Large (Klenow) Fragment Terminal Deoxynucleotidyl Transferase T4 DNA Polymerase Reverse Transcriptases: AMV Reverse Transcriptase M-MLV Reverse Transcriptase.

For certain embodiments, it may be desirable to incorporate a label into the nucleic acid sequences, amplification products, probes or primers. A number of different labels can be used, including but not limited to fluorophores, chromophores, radio-isotopes, enzymatic tags, antibodies, chemiluminescent, electroluminescent, and affinity labels.

Examples of affinity labels contemplated herein, can include, but are not limited to, an antibody, an antibody fragment, a receptor protein, a hormone, biotin, DNP, and any polypeptide/protein molecule that binds to an affinity label.

Examples of enzymatic tags include, but are not limited to, urease, alkaline phosphatase or peroxidase. Colorimetric indicator substrates can be employed with such enzymes to provide a detection means visible to the human eye or spectrophotometrically visible.

The following fluorophores disclosed herein include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

### Gel Electrophoresis

In some embodiments, gel electrophoresis may be used to separate, partially purify or purify a component, identified or contemplated herein using standard methods known in the art.

Separation by electrophoresis is based upon methods known in the art. Samples separated in this manner may be visualized by staining and quantitating, in relative terms, using densitometers which continuously monitor the photometric density of the resulting stain. The electrolyte may be continuous (a single buffer) or discontinuous, where a sample is stacked by means of a buffer discontinuity, before it enters the running gel/running buffer.

### Chromatographic Techniques

Alternatively, chromatographic techniques may be employed to effect separation. There are many kinds of chromatography which may be used for example: adsorption, partition, ion-exchange and molecular sieve, and many specialized techniques for using them including column, paper, thin-layer and gas chromatography.

### Microfluidic Techniques

Microfluidic techniques include separation on a platform such as microcapillaries, designed by ACLARA BioSciences Inc., or the LabChip.™ liquid integrated circuits made by Caliper Technologies Inc. These microfluidic platforms require only nanoliter volumes of sample, in contrast to the microliter volumes required by other separation technologies. Miniaturizing some of the processes involves genetic analysis has been achieved using microfluidic techniques known in the art.

### Nucleic Acid Delivery

### Liposomal Formulations

In certain broad embodiments of the invention, the oligo- or polynucleotides and/or expression vectors may be entrapped in a liposome. Liposomes are vesicular structures characterized by a phospholipid bilayer membrane and an inner aqueous medium. Multilamellar liposomes have multiple lipid layers separated by aqueous medium. The lipid components undergo self rearrangement before the formation of closed structures and entrap water and dissolved solutes between the lipid bilayers (Ghosh and Bachhawat, 1991). Also contemplated are cationic lipid-nucleic acid complexes, such as lipofectamine nucleic acid complexes.

In certain embodiments of the invention, the liposome may be complexed with a hemagglutinating virus (HVJ). This has been shown to facilitate fusion with the cell membrane and promote cell entry of liposome encapsulated DNA (Kaneda et al., 1989). In other embodiments, the liposome may be complexed or employed in conjunction with nuclear non histone chromosomal proteins (HMG 1) (Kato et al., 1991). In yet further embodiments, the liposome may be complexed or employed in conjunction with both HVJ and HMG 1. In that such expression vectors have been successfully employed in transfer and expression of a polynucleotide in vitro and in vivo, then they are applicable for the present invention. Where a bacterial promoter is employed in the DNA construct, it also will be desirable to include within the liposome an appropriate bacterial polymerase.

“Liposome” is a generic term encompassing a variety of single and multilamellar lipid vehicles formed by the generation of enclosed lipid bilayers. Phospholipids are used for preparing the liposomes according to the present invention and can carry a net positive charge, a net negative charge or are neutral. Dicetyl phosphate can be employed to confer a negative charge on the liposomes, and stearylamine can be used to confer a positive charge on the liposomes.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem Behring; dimyristyl phosphatidylglycerol (“DMPG”) and other lipids may be obtained from Avanti Polar Lipids, Inc. (Birmingham, Ala.). Stock solutions of lipids in chloroform, chloroform/methanol or t-butanol can be stored at about 20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

Liposomes used according to embodiments herein can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self associate. For example, when aqueous phases are present both within and without the liposome, the lipid molecules will form a bilayer, known as a lamella, of the arrangement XY YX.

Liposomes within the scope herein can be prepared in accordance with known laboratory techniques.

In certain embodiments, the lipid dioleoylphosphatidylcholine is employed. Nuclease resistant oligonucleotides were mixed with lipids in the presence of excess butanol. The mixture was vortexed before being frozen in an acetone/dry ice bath. The frozen mixture was lyophilized and hydrated with Hepes buffered saline (1 mM Hepes, 10 mM NaCl, pH 7.5) overnight, and then the liposomes were sonicated in a bath type sonicator for 10 to 15 min. The size of the liposomal oligonucleotides typically ranged between 200 300 nm in diameter as determined by the submicron particle sizer autodilute model 370 (Nicomp, Santa Barbara, Calif.).

### Site-Specific Mutagenesis

Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. A primer of about 15 to 30 nucleotides in length can be used, with about 5 to 10 residues on both sides of the junction of the sequence being altered.

In general, the technique of site-specific mutagenesis is well known in the art. As will be appreciated, the technique often employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis can be performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer can then be annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

### Expressed Proteins or Fragments Thereof

Examples of expression systems known to the skilled practitioner in the art include bacteria such as E. coli, yeast such as Pichia pastoris, baculovirus, and mammalian expression systems such as in Cos or CHO cells. A complete gene can be expressed or, alternatively, fragments of the gene encoding portions of polypeptide can be produced.

In certain broad applications herein, a gene sequence encoding a polypeptide is analyzed to detect putative transmembrane sequences. Such sequences are typically very hydrophobic and are readily detected by the use of standard sequence analysis software, such as MacVector (IBI, New Haven, Conn.). The presence of transmembrane sequences is often deleterious when a recombinant protein is synthesized in many expression systems, especially E. coli, as it leads to the production of insoluble aggregates which are difficult to renature into the native conformation of the protein. Deletion of transmembrane sequences typically does not significantly alter the conformation of the remaining protein structure.

To express a recombinant encoded protein or peptide, whether mutant or wild-type, in accordance herein one could prepare an expression vector that includes nucleic acid sequences under the control of, or operatively linked to, one or more promoters. To bring a coding sequence “under the control of” a promoter, one can position the 5′ end of the transcription initiation site of the transcriptional reading frame generally between about 1 and about 50 nucleotides “downstream” (e.g., 3′) of the chosen promoter. The “upstream” promoter stimulates transcription of the DNA and promotes expression of the encoded recombinant protein.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant bacteriophage DNA, plasmid DNA or cosmid DNA expression vectors.

Certain examples of prokaryotic hosts are E. coli strain RR1, E. coli LE392, E. coli B, E. coli X 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is often transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which may be used by the microbial organism for expression of its own proteins.

In addition, phage vectors containing replicon and control sequences that are compatible with the host microorganism may be used as transforming vectors in connection with these hosts. For example, the phage lambda GEMTM-11 may be utilized in making a recombinant phage vector which may be used to transform host cells, such as E. coli LE392.

Further useful vectors include pIN vectors (Inouye et al., 1985); and pGEX vectors, for use in generating glutathione S transferase (GST) soluble fusion proteins for later purification and separation or cleavage. Other suitable fusion proteins are those with B galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

In addition to microorganisms, cultures of cells derived from multicellular organisms may also be used as hosts. In principle, any such cell culture is workable, whether from vertebrate or invertebrate culture.

### Chorismate Super-Pathway and Tyrosine

It is contemplated herein that an amino acid modulating encoding region of microorganisms may be important for increasing production of or tolerance of production of organic acid by the microorganism. In one exemplary method, gene regions encoding tyrosine biosynthetic enzymes and the gene region encoding a repressor for genes involved in tyrosine production can be manipulated in order to increase the tolerance of or production of organic acid by a microorganism.

In certain embodiments, exogenously added tyrosine can be added to a bacterial culture capable of producing 3-HP. In certain particular embodiments, tyrosine concentrations can be about 0.05 mM to about 0.5 mM. In one example, 0.2 mM tyrosine was added to a culture and the increase in 3-HP production was about 35%.

Particular embodiments of the present invention concern oligonucleotides comprising at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 contiguous nucleotides having a sequence selected from the group consisting of SEQ ID NO:1, SEQ ID NO:2, SEQ ID NO:3, SEQ ID NO:4, SEQ ID NO:5, and SEQ ID NO:6

TyrA (this sequence includes 50 bp upstream and downstream for primer design):

SEQ ID NO: 1
TCAGGATCTG AACGGGCAGC TGACGGCTCG CGTGGCTTAA
GAGGTTT
SEQ ID NO: 2:
TTATG GTT GCT GAA TTG ACC GCA TTA CGC GAT CAA ATT
GAT GAA GTC GAT AAA GCG CTG CTG AAT TTA TTA GCG
AAG CGT CTG GAA CTG GTT GCT GAA GTG GGC GAG GTG
AAA AGC CGC TTT GGA CTG CCT ATT TAT GTT CCG GAG
CGC GAG GCA TCT ATG TTG GCC TCG CGT CGT GCA GAG
GCG GAA GCT CTG GGT GTA CCG CCA GAT CTG ATT GAG
GAT GTT TTG CGT CGG GTG ATG CGT GAA TCT TAC TCC
AGT GAA AAC GAC AAA GGA TTT AAA ACA CTT TGT CCG
TCA CTG CGT CCG GTG GTT ATC GTC GGC GGT GGC GGT
CAG ATG GGA CGC CTG TTC GAG AAG ATG CTG ACC CTC
TCG GGT TAT CAG GTG CGG ATT CTG GAG CAA CAT GAC
TGG GAT CGA GCG GCT GAT ATT GTT GCC GAT GCC GGA
ATG GTG ATT GTT AGT GTG CCA ATC CAC GTT ACT GAG
CAA GTT ATT GGC AAA TTA CCG CCT TTA CCG AAA GAT
TGT ATT CTG GTC GAT CTG GCA TCA GTG AAA AAT GGG
CCA TTA CAG GCC ATG CTG GTG GCG CAT GAT GGT CCG
GTG CTG GGG CTA CAC CCG ATG TTC GGT CCG GAC AGC
GGT AGC CTG GCA AAG CAA GTT GTG GTC TGG TGT GAT
GGA CGT AAA CCG GAA GCA TAC CAA TGG TTT CTG GAG
CAA ATT CAG GTC TGG GGC GCT CGG CTG CAT CGT ATT
AGC GCC GTC GAG CAC GAT CAG AAT ATG GCG TTT ATT
CAG GCA CTG CGC CAC TTT GCT ACT TTT GCT TAC GGG
CTG CAC CTG GCA GAA GAA AAT GTT CAG CTT GAG CAA
CTT CTG GCG CTC TCT TCG CCG ATT TAC CGC CTT GAG
CTG GCG ATG GTC GGG CGA CTG TTT GCT CAG GAT CCG
CAG CTT TAT GCC GAC ATC ATT ATG TCG TCA GAG CGT
AAT CTG GCG TTA ATC AAA CGT TAC TAT AAG CGT TTC
GGC GAG GCG ATT GAG TTG CTG GAG CAG GGC GAT AAG
CAG GCG TTT ATT GAC AGT TTC CGC AAG GTG GAG CAC
TGG TTC GGC GAT TAC GCA CAG CGT TTT CAG AGT GAA
AGC CGC GTG TTA TTG CGT CAG GCG AAT GAC AAT CGC
CAG TAA
SEQ ID NO: 3
TAATCCAGTG CCGGATGATT CACATCATCC GGCACCTTTT
CATCAGGTTG
SEQ ID NO: 4
TCAGGATCTG AACGGGCAGC TGACGGCTCG CGTGGCTTAA
GAGGTTTTTA TGGTT GCT GAA TTG ACC GCA TTA CGC GAT
CAA ATT GAT GAA GTC GAT AAA GCG CTG CTG AAT TTA
TTA GCG AAG CGT CTG GAA CTG GTT GCT GAA GTG GGC
GAG GTG AAA AGC CGC TTT GGA CTG CCT ATT TAT GTT
CCG GAG CGC GAG GCA TCT ATG TTG GCC TCG CGT CGT
GCA GAG GCG GAA GCT CTG GGT GTA CCG CCA GAT CTG
ATT GAG GAT GTT TTG CGT CGG GTG ATG CGT GAA TCT
TAC TCC AGT GAA AAC GAC AAA GGA TTT AAA ACA CTT
TGT CCG TCA CTG CGT CCG GTG GTT ATC GTC GGC GGT
GGC GGT CAG ATG GGA CGC CTG TTC GAG AAG ATG CTG
ACC CTC TCG GGT TAT CAG GTG CGG ATT CTG GAG CAA
CAT GAC TGG GAT CGA GCG GCT GAT ATT GTT GCC GAT
GCC GGA ATG GTG ATT GTT AGT GTG CCA ATC CAC GTT
ACT GAG CAA GTT ATT GGC AAA TTA CCG CCT TTA CCG
AAA GAT TGT ATT CTG GTC GAT CTG GCA TCA GTG AAA
AAT GGG CCA TTA CAG GCC ATG CTG GTG GCG CAT GAT
GGT CCG GTG CTG GGG CTA CAC CCG ATG TTC GGT CCG
GAC AGC GGT AGC CTG GCA AAG CAA GTT GTG GTC TGG
TGT GAT GGA CGT AAA CCG GAA GCA TAC CAA TGG TTT
CTG GAG CAA ATT CAG GTC TGG GGC GCT CGG CTG CAT
CGT ATT AGC GCC GTC GAG CAC GAT CAG AAT ATG GCG
TTT ATT CAG GCA CTG CGC CAC TTT GCT ACT TTT GCT
TAC GGG CTG CAC CTG GCA GAA GAA AAT GTT CAG CTT
GAG CAA CTT CTG GCG CTC TCT TCG CCG ATT TAC CGC
CTT GAG CTG GCG ATG GTC GGG CGA CTG TTT GCT CAG
GAT CCG CAG CTT TAT GCC GAC ATC ATT ATG TCG TCA
GAG CGT AAT CTG GCG TTA ATC AAA CGT TAC TAT AAG
CGT TTC GGC GAG GCG ATT GAG TTG CTG GAG CAG GGC
GAT AAG CAG GCG TTT ATT GAC AGT TTC CGC AAG GTG
GAG CAC TGG TTC GGC GAT TAC GCA CAG CGT TTT CAG
AGT GAA AGC CGC GTG TTA TTG CGT CAG GCG AAT GAC
AAT CGC CAG TAA TAATCCAGTG CCGGATGATT CACATCATCC
GGCACCTTTT CATCAGGTTG

SEQ ID NO: 5 TyrR and SEQ ID NO: 6 has the TyrR with the two primers on either end.

SEQ ID NO: 5

ATGCGTCTGG AAGTCTTTTG TGAAGACCGA CTCGGTCTGA
CCCGCGAATT ACTCGATCTA CTCGTGCTAA GAGGCATTGA
TTTACGCGGT ATTGAGATTG ATCCCATTGG GCGAATCTAC
CTCAATTTTG CTGAACTGGA GTTTGAGAGT TTCAGCAGTC
TGATGGCCGA AATACGCCGT ATTGCGGGTG TTACCGATGT
GCGTACTGTC CCGTGGATGC CTTCCGAACG TGAGCATCTG
GCGTTGAGCG CGTTACTGGA GGCGTTGCCT GAACCTGTGC
TCTCTGTCGA TATGAAAAGC AAAGTGGATA TGGCGAACCC
GGCGAGCTGT CAGCTTTTTG GGCAAAAATT GGATCGCCTG
CGCAACCATA CCGCCGCACA ATTGATTAAC GGCTTTAATT
TTTTACGTTG GCTGGAAAGC GAACCGCAAG ATTCGCATAA
CGAGCATGTC GTTATTAATG GGCAGAATTT CCTGATGGAG
ATTACGCCTG TTTATCTTCA GGATGAAAAT GATCAACACG
TCCTGACCGG TGCGGTGGTG ATGTTGCGAT CAACGATTCG
TATGGGCCGC CAGTTGCAAA ATGTCGCCGC CCAGGACGTC
AGCGCCTTCA GTCAAATTGT CGCCGTCAGC CCGAAAATGA
AGCATGTTGT CGAACAGGCG CAGAAACTGG CGATGCTAAG
CGCGCCGCTG CTGATTACGG GTGACACAGG TACAGGTAAA
GATCTCTTTG CCTACGCCTG CCATCAGGCA AGCCCCAGAG
CGGGCAAACC TTACCTGGCG CTGAACTGTG CGTCTATACC
GGAAGATGCG GTCGAGAGTG AACTGTTTGG TCATGCTCCG
GAAGGGAAGA AAGGATTCTT TGAGCAGGCG AACGGTGGTT
CGGTGCTGTT GGATGAAATA GGGGAAATGT CACCACGGAT
GCAGGCGAAA TTACTGCGTT TCCTTAATGA TGGCACTTTC
CGTCGGGTTG GCGAAGACCA TGAGGTGCAT GTCGATGTGC
GGGTGATTTG CGCTACGCAG AAGAATCTGG TCGAACTGGT
GCAAAAAGGC ATGTTCCGTG AAGATCTCTA TTATCGTCTG
AACGTGTTGA CGCTCAATCT GCCGCCGCTA CGTGACTGTC
CGCAGGACAT CATGCCGTTA ACTGAGCTGT TCGTCGCCCG
CTTTGCCGAC GAGCAGGGCG TGCCGCGTCC GAAACTGGCC
GCTGACCTGA ATACTGTACT TACGCGTTAT GCGTGGCCGG
GAAATGTGCG GCAGTTAAAG AACGCTATCT ATCGCGCACT
GACACAACTG GACGGTTATG AGCTGCGTCC ACAGGATATT
TTGTTGCCGG ATTATGACGC CGCAACGGTA GCCGTGGGCG
AAGATGCGAT GGAAGGTTCG CTGGACGAAA TCACCAGCCG
TTTTGAACGC TCGGTATTAA CCCAGCTTTA TCGCAATTAT
CCCAGCACGC GCAAACTGGC AAAACGTCTC GGCGTTTCAC
ATACCGCGAT TGCCAATAAG TTGCGGGAAT ATGGTCTGAG
TCAGAAGAAG AACGAAGAGTAA

The AroF sequence is:

SEQ ID NO: 6

ATGCAAAAAG ACGCGCTGAA TAACGTACAT ATTACCGACG
AACAGGTTTT AATGACTCCG GAACAACTGA AGGCCGCTTT
TCCATTGAGC CTGCAACAAG AAGCCCAGAT TGCTGACTCG
CGTAAAAGCA TTTCAGATAT TATCGCCGGG CGCGATCCTC
GTCTGCTGGT AGTATGTGGT CCTTGTTCCA TTCATGATCC
GGAAACTGCT CTGGAATATG CTCGTCGATT TAAAGCCCTT
GCCGCAGAGG TCAGCGATAG CCTCTATCTG GTAATGCGCG
TCTATTTTGA AAAACCCCGT ACCACTGTCG GCTGGAAAGG
GTTAATTAAC GATCCCCATA TGGATGGCTC TTTTGATGTA
GAAGCCGGGC TGCAGATCGC GCGTAAATTG CTGCTTGAGC
TGGTGAATAT GGGACTGCCA CTGGCGACGG AAGCGTTAGA
TCCGAATAGC CCGCAATACC TGGGCGATCT GTTTAGCTGG
TCAGCAATTG GTGCTCGTAC AACGGAATCG CAAACTCACC
GTGAAATGGC CTCCGGGCTT TCCATGCCGG TTGGTTTTAA
AAACGGCACC GACGGCAGTC TGGCAACAGC AATTAACGCT
ATGCGCGCCG CCGCCCAGCC GCACCGTTTT GTTGGCATTA
ACCAGGCAGG GCAGGTTGCG TTGCTACAAA CTCAGGGGAA
TCCGGACGGC CATGTGATCC TGCGCGGTGG TAAAGCGCCG
AACTATAGCC CTGCGGATGT TGCGCAATGT GAAAAAGAGA
TGGAACAGGC GGGACTGCGC CCGTCTCTGA TGGTAGATTG
CAGCCACGGT AATTCCAATA AAGATTATCG CCGTCAGCCT
GCGGTGGCAG AATCCGTGGT TGCTCAAATC AAAGATGGCA
ATCGCTCAAT TATTGGTCTG ATGATCGAAA GTAATATCCA
CGAGGGCAAT CAGTCTTCCG AGCAACCGCG CAGTGAAATG
AAATACGGTG TATCCGTAAC CGATGCCTGC ATTAGCTGGG
AAATGACCGA TGCCTTGCTG CGTGAAATTC ATCAGGATCT
GAACGGGCAG CTGACGGCTC GCGTGGCTTAA

### EXAMPLES

The following examples are included to demonstrate some embodiments. It should be appreciated by those of skill in the art that the techniques disclosed in the examples which follow represent techniques discovered by the inventors to function well in the practice of embodiments disclosed herein, and thus can be considered to constitute exemplary modes for its practice. However, those of skill in the art should, in light of the present disclosure, appreciate that many changes can be made in certain embodiments which are disclosed and still obtain a like or similar result without departing from the spirit and scope herein.

### Materials and Methods

### Bacteria, Plasmids, and Media

Some methods concern wild-type Escherichia coli K12 (ATCC # 29425) used for the preparation of genomic DNA. Genomic libraries were constructed using the pSMART-LCKAN (Lucigen, Middleton, Wis.). Libraries were introduced into Escherichia coli strain Mach1-T1^{R} (Invitrogen, Carlsbad, Calif.) for selections as previously detailed. Mach1-T1^{R} containing pSMART-LCKAN empty vector were used for all control studies. Growth curves were done in MOPS Minimal Media. In this example, the antibiotic concentration was 20 ug kanamycin/mL.

### Genomic Library Construction

Cultures of the E. coli K12 were cultivated overnight in 500 ml of LB at 37° C. to an optical density of 1.0 measured by absorbance at 600 nm (OD₆₀₀). DNA was extracted using a Genomic DNA Purification kit (e.g. Qiagen) according to manufacturer's instructions. Five samples containing 50 ug of purified genomic DNA were digested using two blunt-cutter restriction enzymes: AluI and RsaI (e.g. Invitrogen). Both enzymes have four base pair recognition sequences and are used in tandem to ensure a random digestion of the genomic DNA. Digestion reactions were carried out with a total volume of 50 uL. The reactions contained 1 unit of Rsa1, 1 unit Alu1, 50 mM Tris-HCl (pH 8.0), and 10 mM MgCl₂ and were incubated at 37° C. for 1, 2, 5, 10, and 15 minutes, respectively. The partially digested DNA was immediately mixed and separated based on size using agarose gel electrophoresis. DNA fragments of 0.5, 1, 2, 4, and greater than 8 kb were excised from the gel and purified with a Gel Extraction Kit (e.g. Qiagen).

The purity of the DNA fragments was quantified using UV absorbance, each with an A260/A280 absorbance ratio of >1.7. Ligation of the purified, fragmented DNA with the pSMART-Kan vectors was performed with the CloneSMART Kit (Lucigen) according to manufacturer's instructions. The ligation product was then electroporated into E. coli 10 GF′ Elite Electrocompetent Cells (Lucigen), plated on LB+kanamycin, and incubated at 37° C. for 24 hours. Dilution cultures with 1/1000 of the original transformation volume were plated on LB+kanamycin in triplicate to determine transformation efficiency and transformant numbers. Dilution plates were done in triplicate to ascertain an accurate count of the number of transformants to ensure a representative genomic library.

Colonies were harvested by gently scraping the plates into TB media. The cultures were immediately resuspended by vortexing, and allocated into 15-1 mL freezer stock cultures with a final glycerol concentration of 15% v/w. The remainder of the culture was pelleted by centrifugation for 15 minutes at 3000 rpm. Plasmid DNA was extracted. To confirm insert sizes and positive transformant numbers, plasmids were isolated from random clones for each library size using for example, a Qiaprep Spin MiniPrep Kit from Qiagen (Valencia, Calif.). Purified plasmids were then analyzed by either PCR or restriction digestion. PCR using the SL1 (SEQ ID NO: 7: 5′-CAG TCC AGT TAC GCT GGA GTC-3′) and SR2 (SEQ ID NO: 8: 5′-GGT CAG GTA TGA TTT AA A TGG TCA GT) primers was performed on eight clones from the 0.5, 1, and 2 kbp insert libraries. Restriction digestions with the enzyme EcorV were carried out for eight clones from the 2, 4, and 8 kbp insert libraries. Inspection by electrophoresis showed that the required number of colonies contained an insert of the expected size for proper representation, chimeras were not present.

### Transformation of Library DNA

Purified plasmid DNA from each library was introduced into MACH1™-T1^{R} (Invitrogen) by electroporation. MACH1™-T1^{R} cultures were made electrocompetent by a standard glycerol wash procedure on ice to a final concentration of 10¹¹ cells/ml (Sanbrook et al.). 1/1000 volume of the original transformations was plated on LB+kanamycin in triplicate to determine transformation efficiency and adequate transformant numbers. The original cultures were combined and diluted to 100 ml with MOPS minimal media+kanamycin and incubated at 37° C. for 6 hours or until reaching an OD₆₀₀ of 0.20.

### Selections

In one exemplary method, four representative genomic libraries were created from E. coli K12 genomic DNA with defined insert sizes of 1, 2, 4, and 8 kb. The transformed library mixture was aliquoted into two 15 mL screw cap tubes with a final concentration of 20 g/L 3-HP (TCI America) neutralized to pH 7 with 10 M NaOH. The cell density of the selection cultures was monitored as they approached a final OD₆₀₀ of 0.3-0.4. The original selection cultures were subsequently used to innoculate another round of 15 mL MOPS minimal media+kanamycin+3-HP as part of a repeated batch selection strategy. Repeated batch cultures containing 3-HP were monitored and inoculated over a 60 hour period to enhance the concentration of clones exhibiting increased growth in the presence of 3-HP. Samples were taken by plating 1 mL of the selected population onto selective plates with each batch. Plasmid DNA was extracted from each sample, then, hybridized to Affymetrix E. coli Antisense GeneChip® arrays (Affymetrix, Santa Clara, Calif.).

### Data Analysis

Data analysis was completed by utilizing a software package, the SCALEs software package, (U.S. patent application Ser. No. 11/231,018 filed Sep. 20, 2005, incorporated herein by reference in its entirety). Fitness contributions from specific genomic elements were calculated from the enrichment of each region as a fraction of the selected population, as was previously described (Lynch, M., Warnecke, T E, Gill, R T, SCALEs: multiscale analysis of library enrichment. Nature Methods, 2007, 4(87-93), incorporated herein by reference in its entirety). Genetic elements and their corresponding fitness were then segregated by metabolic pathway based on their EcoCyc classifications (ecocyc.org). This fitness matrix was used to calculate both pathway fitness (W) and frequency of enrichment found in the selected population.

$$\begin{matrix}{W}_{pathway} = {\sum }_{1}^{n} {W}_{i} \\ frequency = \frac{number of genes from metabolic pathway}{total genes in pathway}\end{matrix}$$

Pathway assignment redundancies were identified by an initial rank ordering of pathway fitness, followed by a specific assignment for genetic elements associated with multiple pathways to the primary pathway identified in the first rank, and subsequent removal of the gene-specific fitness values from the secondary pathways.

### Growth Confirmations

Overnight cultures of Mach1-T1^{R}+pSMART LC-KAN were inoculated into 5 mL LB+kanamycin. Growth curves were constructed by inoculating into 15 mL screw cap tubes containing supplements (Table 1) and 15 mL MOPS Minimal Media+kanamycin+3-HP from overnight culture. Cultures were incubated at 37° C. and optical density was monitored to an OD₆₀₀>0.2. Cultures were then diluted to an exact OD₆₀₀=0.2 and were used to inoculate cultures containing 15 mL MOPS Minimal Media+kanamycin+3-HP (pH=7.0) to an initial optical density of 0.40 in order to minimize effects of growth in stationary phase. Optical density was monitored and recorded over the entire range of microaerobic growth in minimal media, or until a final OD₆₀₀ 0.5-0.6. Growth parameters were evaluated in terms of specific growth, OD₆₀₀ at the culmination of the growth phase (approximately 14 hours), and OD₆₀₀ at conclusion of maximum growth phase and final OD₆₀₀ (24 hrs). To address specific intermediate limitations, associated chorismate pathway supplements were added to final concentrations listed in Table 1.

### Clone Construction

PCR was used to amplify the E. coli K12 genomic DNA corresponding to the aroF-tyrA region with primers designed to include the upstream aroFp promoter and the rho-independent transcriptional terminators. Ligation of the purified, fragmented DNA with the pSMART-kanamycin vectors was performed with the CloneSMART Kit (Lucigen, Middleton, Wis.) according to manufacturer's instructions. The ligation product was then transformed into chemically competent MACH1-T1R (Invitrogen, Carlsbad, Calif.), plated on LB+kanamycin, and incubated at 37° C. for 24 hours. To confirm the insertion of positive transformants, plasmids were isolated from clones using a Qiaprep Spin MiniPrep Kit from Qiagen (Valencia, Calif.) and sequenced (Macrogen, South Korea).

### Example 1

In one exemplary method, a selection was carried out over 8 serial transfer batches with a decreasing gradient of 3-HP over 60 hours. The initial population was comprised of five representative E. coli K12 genomic libraries that were transformed into MACH1-TR and cultured to mid exponential phase corresponding to microaerobic conditions OD₆₀₀˜0.2). Batch transfer times were sustained as variable parameters that were adjusted as needed to avoid a nutrient limited selection environment. Samples were taken at the culmination of each batch in the selection, as described above, and were further analyzed with the SCALEs software in order to decompose the microarray signals into corresponding library clones and calculate relative enrichment of specific regions over time. In this way, genome-wide fitness (ln(Xᵢ/Xᵢ₀)) was measured based on region specific enrichment patterns for the selection in the presence of an industrially relevant organic acid, 3-HP.

FIG. 1 represents plots of genome-wide multiscale analysis from the 3-HP selection. Each peak depicts the signal (fraction of the selected population) represented by the corresponding genomic region. Plots are represented as circles due to the circular chromosome of E. coli, genomic position increases clockwise around each circle with the first and last base pair of the genome at 12 O'Clock. Each plot A, B, C, and D represent the signal associated with the 1000 bp, 2000 bp, 4000 bp and 8000 bp Scales, respectively. The numbers around the circles correspond to genes encoding components of the chorismate super-pathway. These genes were on genomic regions that showed considerable enrichment in the 3-HP selection.

One advantage to the SCALEs approach is the ability to quantitatively track fitness of a clonal population through the duration of selection. Fitness of individual clones can then be segmented by gene and further categorized by pathway, creating a genome-wide spectrum of pathway fitness contributing to overall 3-HP tolerance. Using this method, several key metabolic pathways have been identified that include the majority of clones contributing fitness to the system (FIG. 2). FIG. 2 represents pathway fitness results for the top 7 pathways contributing to overall fitness. The chorismate super-pathway has been recognized as both the largest contribution to overall fitness as well as the highest frequency of genetic elements contained in the selected population, with 19 genetic elements identified in the top 10% of the population exhibiting increased fitness (FIG. 3A). FIG. 3A represents the chorismate super-pathway of E. coli. Enrichment levels for genes found in top 10% of clones are highlighted. Additionally, 33 genes involved in the chorismate super-pathway exhibited significant fitness gains and all 57 genes showed some degree of enrichment throughout the selection. Thus, clones containing genetic elements encoding necessary enzymes downstream of chorismate demonstrated significant fitness increases in the presence of inhibitory levels of 3-HP. This finding indicates that the observed growth inhibition associated with a culture in the presence of 3-HP is the result of an interruption of the chorismate biosynthetic pathway.

FIG. 3A represents a schematic of the chorismate super-pathway. Intermediates are labeled, or otherwise indicated in the junction of arrows. Gene names encoding enzymatic function (arrows) are written next to the corresponding arrows. Negative feedback inhibition of products or intermediates in the pathway are shown as grey arrows.

### Chorismate Pathway Inhibition

To confirm these findings, the culture medium was supplemented with products synthesized downstream of chorismate. The addition of each product individually stimulated growth, further confirming that the inhibition is occurring at, or prior to synthesis of chorismate (FIG. 3B). FIG. 3B represents growth confirmations: addition of products downstream of chorismate partially alleviate growth inhibition confirmed by increased specific growth (black) and increased OD₆₀₀ at the culmination of the growth phase (grey). However, increasing the supplementation of several downstream products (tyrosine, phenylalanine, tryptophan) results in feedback inhibition of the first committed step to the chorismate super-pathway and will therefore reduce formation of other downstream products including ubiquinone, meniquinone, and tetrahydrofolate and limit the associated growth benefits. Here, the addition of chorismate derivatives to the growth medium in the absence of 3-HP had little to no beneficial effect on specific growth or final cell density, further confirming that the supplementation is 3-HP dependant. To further investigate the observed inhibition, the central chorismate pathway intermediate, shikimate was supplied extracellularly and resulted in a 20% increase in specific growth (FIG. 3B).

FIG. 3B represents exemplary methods for illustrating fitness (increased growth rate in the presence of 3-HP) associated with increased copy of genes in the chorismate super-pathway.

Furthermore, in the first step of the chorismate pathway, erythrose-4-phosphate (E-4-P) reacts with phosphoenolpyruvate (PEP) to form 3-deoxy-D-arbino-heptulosonate-7-phosphate. E-4-P is required for several key pathways, including the non-oxidative branch of the pentose phosphate pathway and the biosynthesis of pyridoxal-5′-phosphate (vitamin B6). One finding implies that the E-4-P pool is not limited and that an inhibition most likely occurs between the formation of E-4-P and shikimate. In another experiment, ribose, histidine, and nucleotides were added to the growth media individually. These molecules are byproducts of the histidine, purine, and pyrimidine biosynthesis super-pathway (PRPP), which also contributes significant fitness to the pathway analysis (FIG. 3B)

### Disrupted Feedback Inhibition

By use of the SCALEs methodology, a number of genetic targets for alleviating growth inhibition in the presence of 3-HP have been identified. Specifically, as depicted in FIG. 2, increased copy of the tyrA-aroF operon resulted in significant enrichment throughout the selections, making this genetic region an attractive target. A clone was constructed containing the tyrA-aroF operon and was cultured in the presence of 20 g/L 3-HP. Increased copy of this region partially alleviated growth inhibition, conferring a 15% increase in specific growth. While this region showed significant fitness gains, the associated increase in tyrosine and phenylalanine production inhibited the first step in the chorismate pathway. One method to bypass this inherent control was obtaining an inducible feedback resistant aroH mutant that will increase the conversion of E-4-P while maintaining activity in the presence of increasing pools of downstream products, thus alleviating growth inhibition due to impaired synthesis of necessary byproducts of the chorismate pathway. Growth of the aroH mutant in the presence of 20 g/L 3-HP resulted in a significant increase in specific growth. In addition, the 24 hour minimum inhibitory concentration of 3-HP (the minimum concentration to stop visible growth at 24 hours) in M9 minimal media increased from 25 g/L for a vector control to 40 g/L for an E. coli clone expressing this aroH mutant. In certain embodiments herein, it is contemplated that the aroH mutant can be of use alone, or in combination with other genetic manipulations or selection to increase tolerance of 3-HP production in microorganisms.

This growth inhibition described above can affect downstream aromatic acids, tyrosine, phenylalanine, and tryptophan. In accordance with this growth inhibition, increased pools of these amino acids decreases the activity of the DAHPS isozymes corresponding to the first committed step of the chorismate super-pathway. This example indicates that increased tolerance is not specific to increasing concentrations of each intermediate pool but can be achieved by modulation of the pools. In one exemplary method, supplementation of the growth medium with phenylalanine had detrimental effect on specific growth in the presence of 3-HP while the addition of tyrosine has a beneficial effect. This illustrates the concept that optimal 3-HP tolerance could be achieved by modulating the product concentrations by lowering the phenylalanine pools while simultaneously increasing the tyrosine pools to allow for optimal activity of the DAHPS enzyme. One exemplary embodiment concerns modulating product concentrations of the chorismate super-pathway by lowering the phenylalanine in combination with increasing tyrosine levels to allow for optimal activity of the DAHPS enzyme.

FIG. 4 represents growth confirmation using exemplary components downstream of chorismate in the chorismate super-pathway for reducing growth inhibition. Increased specific growth is illustrated in black while increased OD₆₀₀ at the culmination of the growth phase is illustrated in grey. It is contemplated herein that one or more downstream products of the chorismate super-pathway can be used to increase 3-HP tolerance in a microorganism. In accordance with these uses, one or more downstream products may be supplemented to cultures of microorganisms.

3-HP composition obtained, for example, from TCI America for initial library selections and all subsequent growth confirmations can contain variable amounts of acrylic acid contamination. A minimum inhibitory concentration of acrylic acid for E. coli Mach1 grown in minimal media was determined to be around 0.6 g/L. In support that the tolerance mechanism specific to the chorismate super-pathway are exclusive to 3-HP toxicity, the minimum inhibitory concentrations of acrylic acid was determined to be 0.6 g/L for E. coli Mach1 grown in minimal media supplemented with addition of shikimate or homocysteine. Additionally, the minimal inhibitory concentration was determined to be 0.6 g/L for the feedback resistant aroH mutants grown in minimal media. This data is in support that increasing concentrations of any intermediate involved in the chorismate super-pathway increases tolerance specific to 3-HP toxicity and is not affected by acrylic acid contamination of 3-HP compositions.

In examples described herein, the addition of downstream products from chorismate to the growth medium increased specific growth, confirming that inhibition of organic acid production or growth can be due to limitations of chorismate-related amino acids and essential vitamins. Supplemental shikimate also caused a dramatic increase in growth, indicating that inhibition is occurring prior to shikimate in the chorismate biosynthesis pathway. Further studies suggest that inhibition lies between the formation of erythrose-4-phosphate and shikimate. The findings presented above greatly assist in overcoming the challenge of creating a 3-HP tolerant strain for use as a recombinant host.

In examples described herein, changes in expression or addition of genetic elements containing genes in the chorismate super-pathway demonstrate an increased specific growth in the presence of 3-HP thus increasing tolerance for 3-HP production.

TABLE 1
Media supplementation and associated growth effects compared with
empty vector control.

| | | % Specific |
|---|---|---|
| Supplementation | Concentration | Growth Increase |
| None | N/A | 0 |
| Tyrosine | 0.4 mM | 9 |
| Phenylalanine | 0.4 mM | 10 |
| Tryptophan | 0.1 mM | 1 |
| para-hydroxybenzoate | 0.2 mM | 10 |
| para-aminbenzoate | 0.2 mM | 17 |
| 2,3-dihidroxybenzoate | 0.2 mM | 9 |
| Combination* | | 16 |
| Shikimate | 0.4 mM | 20 |
| Pyridoxine | 2 mM | 0 |

*Combination includes: tyrosine, phenylalanine, para-hydroxybenzoate, para-aminobenzoate and 2,3 dihydroxybenzoate at above concentrations.

All of the COMPOSITIONS and/or METHODS and/or APPARATUS disclosed and claimed herein can be made and executed without undue experimentation in light of the present disclosure. While the compositions and methods have been described in terms of preferred embodiments, it will be apparent to those of skill in the art that variation may be applied to the COMPOSITIONS and/or METHODS and/or APPARATUS and in the steps or in the sequence of steps of the method described herein without departing from the concept, spirit and scope of herein. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept as defined by the appended claims.

1-31. (canceled)

32. A genetically modified microorganism that produces 3-HP, wherein said genetically modified microorganism comprises a genetic modification in its chorismate super-pathway.

33. The genetically modified microorganism of claim 1, wherein the genetically modified microorganism has a tolerance to 3-HP that is at least 1.2-fold greater than the tolerance to 3-HP of the wild-type of said microorganism.

34. The genetically modified microorganism of claim 1, wherein said genetically modified microorganism exhibits increased growth in the presence of 20 g/L of 3-HP.

35. The genetically modified microorganism of claim 3, wherein the genetic modification is in a gene selected from the group consisting of: tyrA, aroA, aroB, aroC, aroD, aroE, aroF, aroG, aroH, aroK, aroL, aspC, entA, entB, entC, entD, entE, entF, folA, folB, folC, folD, folE, folK, folP, menA, menB, menC, menD, menE, menF, pabA, pabB, pabC, pheA, purN, trpA, trpB, trpC, trpD, trpE, tyrB, ubiA, ubiB, ubiC, ubiD, ubiE, ubiF, ubiG, ubiH, ubiX, and ydiB.

36. The genetically modified microorganism of claim 3, wherein the genetic modification is at least one of: a genetic insertion, a disruption or a removal of existing genetic material, a mutation of genetic material, and adding a vector to introduce new genetic material.

37. The genetically modified microorganism of claim 1, wherein said genetically modified microorganism is E. coli.

38. A method for increasing tolerance to 3-HP in a microorganism that produces 3-HP comprising: modifying one or more genes of the chorismate super-pathway in said microorganism.

39. The method of claim 7 wherein said one or more genes are selected from the group consisting of: tyrA, aroA, aroB, aroC, aroD, aroE, aroF, aroG, aroH, aroK, aroL, aspC, entA, entB, entC, entD, entE, entF, folA, folB, folC, folD, folE, folK, folP, menA, menB, menC, menD, menE, menF, pabA, pabB, pabC, pheA, purN, trpA, trpB, trpC, trpD, trpE, tyrB, ubiA, ubiB, ubiC, ubiD, ubiE, ubiF, ubiG, ubiH, ubiX, and ydiB.

40. The method of claim 7, wherein said modifying comprises at least one of: adding a vector to introduce new genetic material, performing a genetic insertion, disrupting or removing existing genetic material, and mutating existing genetic material.

41. The method of claim 7, wherein said microorganism exhibits increased growth in the presence of at least 20 g/L of 3-HP.

42. The method of claim 7, wherein said microorganism has a tolerance to 3-HP that is at least 1.2-fold greater than the tolerance to 3-HP of a wild-type microorganism, wherein the one or more genes of the chorismate super-pathway of the wild-type microorganism have not been modified.

43. The method of claim 10, wherein said microorganism has a tolerance to 3-HP that is at least 1.2-fold greater than the tolerance to 3-HP of a wild-type microorganism, wherein the one or more genes of the chorismate super-pathway of the wild-type microorganism have not been modified.

44. A microorganism made by the method of claim 7.

45. A microorganism made by the method of claim 8.

46. A microorganism made by the method of claim 9.

47. A microorganism made by the method of claim 10.

48. A method of increasing the production and/or tolerance for production of 3-HP by a microorganism comprising:
a) obtaining one or more compounds that is a member of the chorismate super-pathway, and
b) introducing the one or more compounds to a culture of the microorganism.

49. The method of claim 17, wherein the one or more compounds is selected from chorismate, tyrosine, phenylalanine, tryptophan, folate, ubiquinone, meniquinone, shikimate, D-Erythrose-4-phosphate, 3-deoxy-D-arabino-heptulosonate-7-phosphate, 3-dehydroquinate, 3-dehydro-shikimate, shikimate-3-phosphate, 5-enolpyruvyl-shikimate-3-phosphate, isochorismate, prephenate, phenylpyruvate, para-hydroxyphenylpyruvate, 2,3-dihydro-2,3-dihydroxybenzoate, 2,3-dihydroxybenzoate, enterobactin, 2-succinyl-6-hydroxy-2,4-cyclohexadiene-1-carboxylate, o-succinybenzoate, o-succinylbenzoyl-coA, 1,4-dihydroxy-2-napthoate, menaquinone, anthranilate, N-(5′-phosphoribosyl)-anthranilate, 1-(o-carboxyphenylamino)-1′-deoxyribulose-5′-phosphate, indole-3-glycerol-phosphate, indole, 4-amino-4-deoxychorismate, para-aminobenzoate, 7,8-dihydropteroate, 7,8-dihydrofolate, tetrahydrofolate, 4-hydroxybenzoate, 3-octaprenyl-4-hydroxybenzoate, 2-octaprenylphenol, 2,2-octaprenyl-6-hydroxyphenol, 2-octaprenyl-6-methoxyphenol, 2-octaprenyl-6-methoxy-1,4-benzoquinone, 2-octaprenyl-3-methyl-6-methoxy-1,4-benzoquinone, 3-demethylubiquinone-8, ubiquinone-8,3-deoxy-D-arbino-heptulosonate-7-phosphate synthase (DAHPS) isozymes, or mixtures thereof.

50. The method of claim 17, wherein said introducing the one or more compounds comprises introducing at least one compound resulting in at least ten percent increased specific growth of the microorganism in the presence of 3-HP.

51. The method of claim 17, wherein said introducing the one or more compounds comprises introducing more than one compound resulting in an increased specific growth in the presence of 3-HP exceeding 15 percent.
